(19) United States
(12) Patent Application Publication
(10) Pub. No.: US 2019/0008539 A1
(43) Pub. Date: Jan. 10, 2019

(54) DRILLING AND SHAVING ASSEMBLY FOR TISSUE

(71) Applicant: MAXPOL Co., Ltd., Uiwang-si (KR)

(72) Inventors: CHANG YOUNG KIM, Gwangju-si (KR); HAE YOUNG KIM, Gwangju-si (KR); KWANG SUN KIM, Siheung-si (KR); JONG KYUNG JEON, Suwon-si (KR)

(73) Assignee: MAXPOL Co., Ltd., Uiwang-si (KR)

(21) Appl. No.: 16/021,030

(22) Filed: Jun. 28, 2018

(30) Foreign Application Priority Data
Jul. 5, 2017 (KR) 10-2017-0085227

(51) Int. Cl. A61B 17/32; A61B 1/00; A61B 17/16; A61B 17/3207

(52) U.S. Cl. CPC A61B 17/32002; A61B 1/00087; A61B 17/1622; A61B 17/320783; A61B 2017/320032; A61B 17/1633

(57) ABSTRACT

Provided is an assembly for dissecting a tissue including a tissue dissector and a bending device. The tissue dissector includes an outer tube inserted into the human body; an inner tube inserted into the outer tube so that a front end protrudes from a front end of the outer tube; a cutting head located at the front end of the inner tube; a cable connected to a rear end of the cutting head; a driving shaft connected to a rear end of the cable; and a drive motor for applying a rotational force to the driving shaft.

The bending device includes a fitting portion to which the outer tube is fitted; a rotation lever rotatably coupled to a rear end of the fitting portion; a first roller fixed to an end of the fitting portion so as to be in close contact with a front end of the inner tube; a second roller which is fixed to the rotation lever so as to face the first roller with the inner tube interposed therebetween and applies a bending force to the inner tube while rotating about the first roller when the rotation lever rotates; an angle adjusting groove provided on a rotation path of the end of the rotation lever when the rotation lever is rotated to one side of the fitting portion; and an angle adjusting pin which is provided in the angle adjusting groove so as to interfere with an end of the rotating rotation lever so as to restrict a rotation angle of the rotation lever.

A semicircular groove for maximizing the adhesion with the inner tube may be provided on an outer circumferential surface of the first roller and the second roller.

### CROSS-REFERENCE TO RELATED APPLICATIONS

This application claims the benefit of the Korean Patent Application No. 10-2017-0085227, filed on Jul. 5, 2017, which is hereby incorporated by reference as if fully set forth herein.

### TECHNICAL FIELD

The present invention relates to a tissue dissector capable of easily dissecting a tissue while being inserted into a body and an assembly for dissecting a tissue capable of rapidly and accurately bending the tissue dissector at a predetermined angle without damaging the tissue dissector.

### BACKGROUND ART

In general, endoscopy is a surgical method of performing treatment of a portion to be treated by operating a small hole of about 1 cm in the body of a patient, inserting an endoscopy catheter into the hole, passing a required surgical tool through the endoscope catheter, and is widely used in neurosurgery and orthopedic surgery.

There are many kinds of tissue dissectors, and for example, US Patent Publication No. US2012/0316591(Arthroscopic Shaver Handpiece with Modular attachments) is disclosed.

The conventional tissue dissector described above includes an outer fixing member having a cutting window formed therein, a shaver blade having a rotation member (hereinafter referred to as a “cutting head”) axially rotatable in the inner side of the outer fixing member and having a self-cutting window, and a handpiece having a collet for receiving and fixing the shaver blade, and a motor for axially rotating the cutting head of the shaver blade inserted to the collet. In addition, the handpiece is provided with a suction tube for sucking tissue fragments cut out during a surgical procedure and discharging the tissue fragments out of the human body.

However, in the conventional tissue dissector described above, since the outer fixing member is formed in a straight shape, it is very difficult to approach the cutting head to a target point by avoiding obstacles or dissect the tissue at an optimal angle.

In addition, in the conventional tissue dissector, since the shaver blade is detachably attached to the handpiece, there is a problem in that vacuum pressure may be leaked to a gap of a connection portion between the suction tube of the handpiece and the cutting head.

If the vacuum pressure is leaked, the suction is not normally performed, and the possibility that some of the cut tissue fragments will be lost in the body is very increased, and if the lost tissue fragments are cancer cells, there is a serious problem causing a fatal result to the human body.

### DISCLOSURE

### Technical Problem

In order to solve the problems and defects in the related art, an object of the present invention is to provide a tissue dissector and a bending apparatus therefor capable of approaching the cutting head to a target point by avoiding obstacles or dissecting the tissue at an optimal angle and preventing suction pressure from being lowered.

The objects of the present invention are not limited to the aforementioned objects, and other objects, which are not mentioned above, will be apparent to a person having ordinary skill in the art from the following description.

### Technical Solution

In order to achieve the object, the present invention provides a tissue dissector including: an outer tube inserted into the human body; an inner tube inserted into the outer tube so that a front end protrudes from a front end of the outer tube; a cutting head located at the front end of the inner tube; a cable connected to a rear end of the cutting head; a driving shaft connected to a rear end of the cable; and a drive motor for applying a rotational force to the driving shaft.

Further, a suction tube connected to a rear end of the outer tube so as to collect the dissected tissue by introducing the tissue dissected by the cutting head through the front end of the outer tube and a fluid supply tube discharging a fluid to the front end of the outer tube may be connected to the rear end of the outer tube.

In this case, the tissue dissector may further include a closed type second housing which accommodates a rear end of the inner tube passing through the rear end of the outer tube, a rear end of the driving shaft passing through the rear end of the inner tube, and the rotation shaft of the driving motor, in which the fluid supply tube and the suction tube may non-detachably communicate with the outer surface inside the sealed second housing.

In addition, a bending device for the tissue dissector of the present invention may include a fitting portion to which the outer tube is fitted; a handle coupled rotatably to an end of the fitting portion; a first roller fixed to the end of the fitting portion so as to be in close contact with a front end of the inner tube of the tissue dissector; and a second roller which is fixed to the rotation lever so as to face the first roller with the inner tube interposed therebetween and applies a bending force to the inner tube while rotating about the first roller when the rotation lever rotates.

### Advantageous Effects

According to the tissue dissector of the present invention configured above, since the cutting head is connected to the driving shaft via a flexible cable to be normally rotated even though the inner tube is bent, it is possible to very precisely cut the tissue by approaching the tissues located at angles which are difficult to be cut and avoiding the obstacles.

In addition, when a suction tube and a fluid supply tube are provided like the related art, it is possible to greatly improve safety of surgery by introducing and collecting the tissue dissected by the cutting head through the front end of the outer tube or supplying a liquid medicine required for the surgery to the inside of the human body.

Further, since the fluid supply tube and the suction tube non-detachably communicate with the outer tube inside the sealed second housing, it is possible to stably maintain a fluid supply pressure and a suction pressure without deteriorating.

In addition, since the bending device for the tissue dissector of the present invention includes a first roller and a second roller with the inner tube of the tissue dissector interposed therebetween, the inner tube is bent while the second roller rotates based on the first roller with the inner tube interposed therebetween only when the rotation lever rotates, and thus the inner tube is very easily bent and used at a surgery place without dissembling the tissue dissector unlike the related art. As a result, it is possible to greatly improve the accuracy of the surgery.

The effects of the present invention are not limited to the aforementioned effects, and other effects, which are not mentioned above, will be apparent to a person having ordinary skill in the art from the description of claims.

### DESCRIPTION OF DRAWINGS

FIG. 1 is a side view of a tissue dissector according to an exemplary embodiment of the present invention;

FIG. 2 is a cross-sectional view of the tissue dissector according to the exemplary embodiment of the present invention;

FIG. 3 is a detailed view of a part ‘A’ of FIG. 2;

FIG. 4 is a detailed view of a part B′ of FIG. 2;

FIG. 5 is a detailed view of a second housing of FIG. 2;

FIG. 6 is a view showing a state in which an internal tube is bent, as the part ‘A’ of FIG. 2;

FIG. 7 is a view showing another example of a cutting head, as the part ‘A’ of FIG. 2;

FIG. 8 is a view showing another example of a cutting window which is applied to the tissue dissector according to the exemplary embodiment of the present invention;

FIG. 9 is a rear perspective view of a tissue dissector with a handle according to another exemplary embodiment of the present invention;

FIG. 10 is a used state side view of a bending device for the tissue dissector according to the exemplary embodiment of the present invention;

FIG. 11 is a cross-sectional view of FIG. 10.

FIG. 12 is a front perspective cross-sectional view of a bending device for the tissue dissector according to the exemplary embodiment of the present invention; and

FIG. 13 is a view showing a state in which an inner tube is bent while the handle is rotated.

### MODES OF THE INVENTION

Hereinafter, preferred embodiments of the present invention in which the above objects can be specifically implemented will be described in detail with reference to the accompanying drawings. When describing the embodiments, like names and reference numerals designate like components and as a result, the additional description will be omitted. First, configurations and functions according to a preferred embodiment of the present invention will be described with reference to the accompanying drawings.

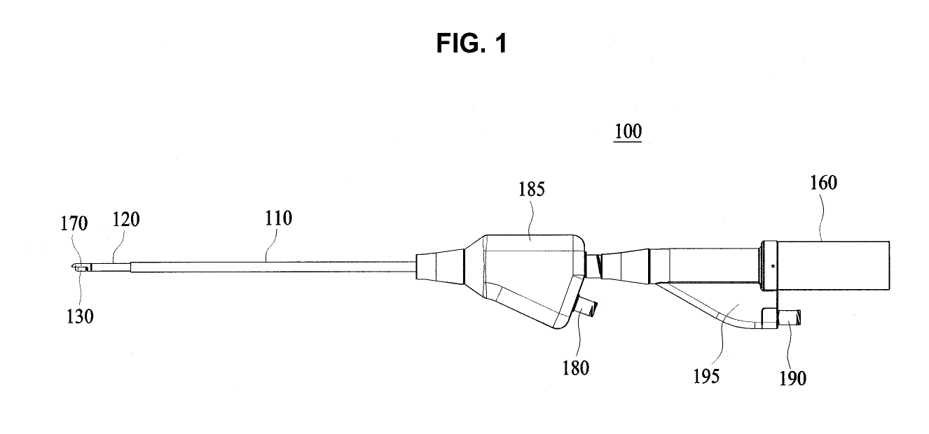

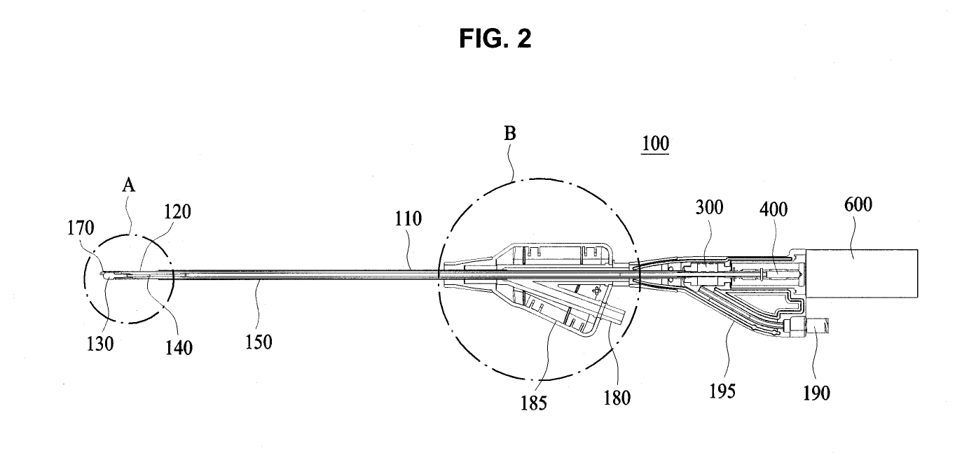

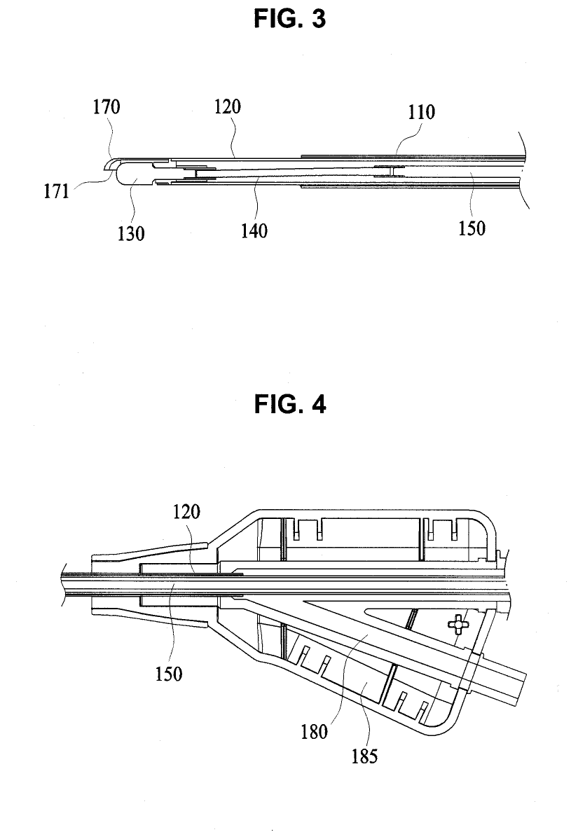

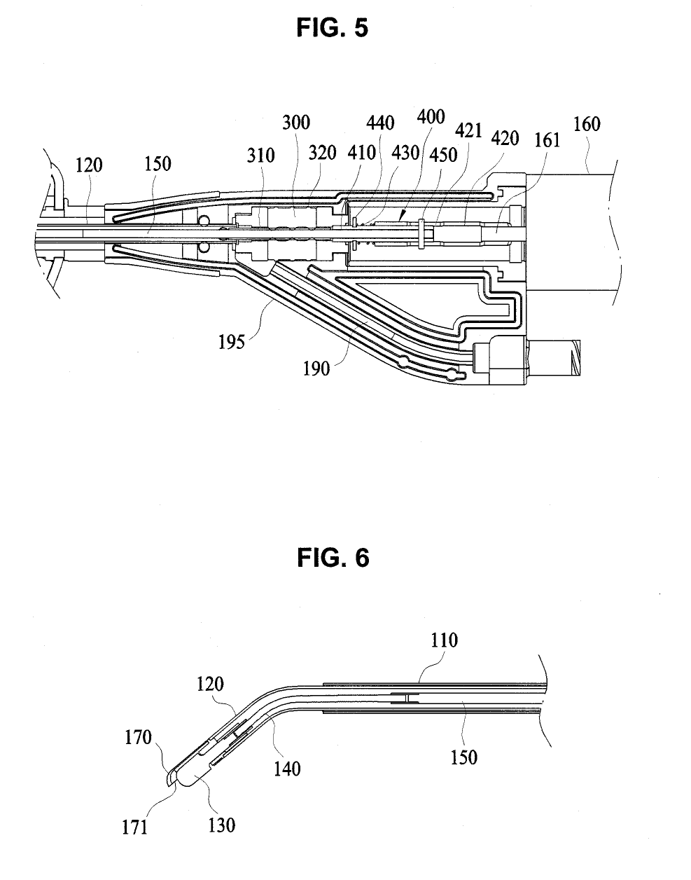

FIG. 1 is a side view of a tissue dissector according to an exemplary embodiment of the present invention, FIG. 2 is a cross-sectional view of the tissue dissector according to the exemplary embodiment of the present invention, FIG. 3 is a detailed view of a part ‘A’ of FIG. 2, FIG. 4 is a detailed view of a part B′ of FIG. 2, and FIG. 5 is a detailed view of a second housing of FIG. 2. As shown in the drawings, a tissue dissector 100 according to the exemplary embodiment of the present invention is configured by an outer tube 110, an inner tube 120 inserted to the outer tube 110, a cutting head 130 provided at a front end of the inner tube 120, a cable 140 and a driving shaft 150 sequentially connected to the cutting head 130, and a driving motor 160 rotating the driving shaft 150.

The outer tube 110 is inserted into the human body as a straight tube having both open ends. Accordingly, the outer tube 110 needs to be made of a material that is harmless to the human body and has sufficient strength not to be bent or broken during insertion so as to be inserted at a precise target point. The inner tube 120 is inserted into the outer tube 110.

The inner tube 120 is inserted into the outer tube 110 so that the front end thereof protrudes from the front end of the outer tube 110. The cutting head 130 is provided at the front end of the inner tube 120. The inner tube 120 needs to be made of a material that is harmless to the human body and has sufficient strength not to be bent or broken during insertion so as to be inserted at a precise target point.

The cutting head 130 is fitted to the front end of the inner tube 120 so as to be rotatable, and it is preferable that the cutting head 130 is fabricated to have a larger diameter than the inner tube 120 so as to be easily in contact with an abnormal tissue to be dissected.

A diamond coating layer may be formed on the surface of the cutting head 130 to improve cutting performance. In this case, it is preferable that the cutting head 130 during cutting is easily distinguished by mixing a material reflected to light at the time of a diamond coating process or thermo ink which is colored with a specific color when the temperature of the cutting head 130 is increased during cutting, with a diamond coating solution, thereby performing precise cutting.

The cable 140 is connected to a rear end of the cutting head 130 and the driving shaft 150 is connected to the rear end of the cable 140. The driving shaft 150 is connected to a rotation shaft 161 of the driving motor 160 provided at the rear end of the driving outer tube 110 to receive a rotational force.

Accordingly, when the driving motor 160 is operated, the rotational force of the driving shaft 150 is transmitted to the cutting head 130 through the cable 140 while the driving shaft 150 is rotated to rotate the cutting head 130. When the cutting head 130 rotates, the tissues contacting the cutting head 130 are dissected. At this time, the outer tube 110 and the inner tube 120, which do not receive the rotational force from the driving motor 160, are not rotated.

On the other hand, as shown in FIG. 6, when a tissue located behind the obstacle is to be dissected, the inner tube 120 needs to be bent at an appropriate angle so as to bring the cutting head 130 close to the tissue to be dissected.

In this case, since the cable 140 has a flexible characteristic, the rotational force of the driving shaft 150 is very smoothly transmitted to the cutting head 130 even if the inner tube 120 is bent.

In addition, a cutting window 170 may be provided at an end of the inner tube 120 to allow the cutting head 130 to have a cutting force only in a specific direction. The cutting window 170 has an opening 171 for exposing the cutting head 130 to the outside in only the specific direction and covers the cutting head 130 with a predetermined interval. Accordingly, the cutting head 130 may precisely dissect only the target tissue through the cutting window 170.

According to the present invention, a suction tube 180 may be connected to a rear end of the outer tube 110. The suction tube 180 introduces the tissue dissected by the cutting head 130 through the front end of the outer tube 110 and discharges the dissected tissue to the outside to prevent an accident that a part of the dissected tissue is left inside the human body.

Meanwhile, it is preferable that a first housing 185 is provided on the outside of the suction tube 180 in order to easily assemble the suction tube and stably connect the suction tube 180 to the outer tube 110.

In addition, a fluid supply tube 190 may be connected to the front end of the outer tube 110 at the rear end of the outer tube 110 to supply liquid medicines required for the surgical procedure, such as a saline solution or an epinephrine mixture, to a surgical site. For example, the saline solution may reduce the risk of hemorrhage, heat damage and perforation of surrounding tissues, or easily remove an abnormal tissue to be dissected through uplifting, and the epinephrine mixture alleviates postoperative pain.

In addition, for convenience of assembling and stable connection to the outer tube 110 of the fluid supply tube 190, it is preferable to further include a closed-type second housing 195 which accommodates the rear end of the inner tube 120 passing through the rear end of the outer tube 110, the rear end of the driving shaft 150 passing through the rear end of the inner tube 120, and the rotation shaft 161 of the driving motor 160.

As such, when the second housing 195 is provided, the fluid supply tube 190 may be configured to communicate with the rear end of the outer tube 110 through the second housing 195.

In the exemplary embodiment, the suction tube 180 is configured to communicate with the second housing 195 located at the rear side while covering the inner tube 120 passing through the rear end of the outer tube 110 and is supplied to the fluid tube 190 to introduce the fluid supplied by the fluid supply tube 190 to the rear end of the outer tube 110 via the suction tube 180 after entering the rear end of the suction tube 180 through the front end of the second housing 195.

Meanwhile, a shielding member 300 fitting the end of the inner tube 120 to the discharged driving shaft 150 is provided to prevent the fluid discharged from the fluid supply tube 190 from being introduced into the second housing 195. An inner surface 310 of the shielding member 300 closely contacts the driving shaft 150 and an outer surface 320 closely contacts an inner wall surface of the second housing 195.

When the driving shaft 150 receives an impact from the driving motor 160 and moves forward and backward, it is difficult to precisely remove the tissue, and in some cases, the drive shaft 160 may be fatal to the human body. It is preferable that a shock absorbing unit 400 is further included between the driving motor 160 and the driving shaft 150 to prevent the impact from the driving motor 160 from being transferred to the driving shaft 150.

The shock absorbing unit 400 includes a fixing tube 410 which is fixed so as not to move forward and backward in the second housing 195 while covering the rear end of the driving shaft 150, a sliding tube 420 having a front end slidably fitted to the outside of the rear end of the fixing tube 410 and a rear end coupled with the rotation shaft 161 of the driving motor 160, and an elastic member 430 supported to the front end of the sliding tube 420. In addition, a snap ring 440 for holding the front end of the elastic member 430 is fitted to the fixing tube 410.

Accordingly, when an impact is generated from the rotation shaft 161 of the driving motor 160, the sliding tube 420 moves forward or backward due to the impact, and at this time, the elastic member 430 absorbs the impact while being contracted or stretched. Therefore, the impact to the driving shaft 150 is minimized.

On the other hand, on both sides of the sliding tube 420, long holes 421 facing each other are formed so that the fixing tube 410 and the sliding tube 420 are not separated from each other. The long holes 421 are formed forward and backward, and the fixing pin 450, which is engaged with the long holes 421, is coupled to the fixing tube 410. Due to the fixing pin 450, the sliding tube 420 is restricted from moving forward and backward over a certain range.

It is preferable that the fixing pin 450 is configured to pass through the driving shaft 150 so that the fixing tube 410 and the driving shaft 150 are not separated from each other.

The tissue stripper 100 of the present invention configured above is used as follows.

First, when the cutting head 130 approaches the abnormal tissue to be dissected by inserting the outer tube 110 is to the dissected lesion, the driving motor 160 is operated. When the driving motor 160 is operated, the cutting head 130 starts to rotate. In this case, if necessary, the liquid medicine required for the surgical procedure, such as a saline solution or an epinephrine mixture may be supplied to the surgical site by the fluid supply tube 190.

When the cutting head 130 is coated with a reflective material or thermal ink, it is easy to determine the position and cutting state of the cutting head 130 through reflected light or the color of the changed cutting head 130, thereby very precisely cu the abnormal tissue.

The abnormal tissue is discharged through the suction tube 180 to the outside immediately after cutting. At this time, since the suction tube 180 shares a flow path with the fluid supply tube 190, the suction tube 180 can operate only when the fluid supply is stopped.

Since the fluid supply tube 190 and the suction tube 180 are configured to non-detachably communicate with the outer tube 110 inside the sealed second housing 195, the fluid supply pressure and the suction pressure are always stably maintained.

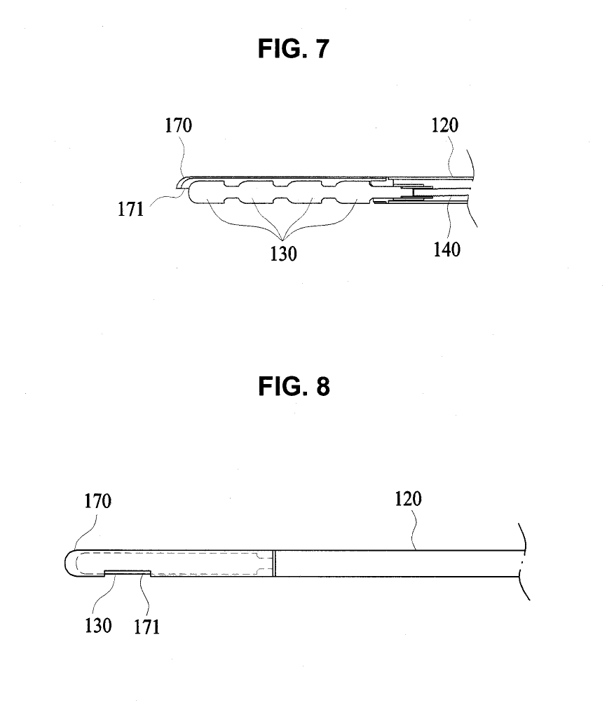

Meanwhile, as shown in FIG. 7, a plurality of cutting heads 130 may be provided at predetermined intervals in a vertical direction so that the cutting head 130 can quickly cut a wide area.

As shown in FIG. 8, the cutting window 170 may be configured to be exposed to the outside only in a specific direction with respect to a partial section of the cutting head 130. In this case, it is preferable that the cutting head 130 is fabricated to be elongated in the vertical direction.

Accordingly, a user appropriately selects a cutting window 170 in which the cutting head 130 is opened at a position of an abnormal tissue to be doubly dissected from a plurality of cutting windows 170 having different positions of the openings 171 and uses the selected cutting window to be coupled to the inner tube 120, thereby more easily and accurately dissecting the tissue.

Meanwhile, the plurality of openings 171 may be formed. In this case, abnormal tissues existing at a plurality of positions can be simultaneously dissected quickly.

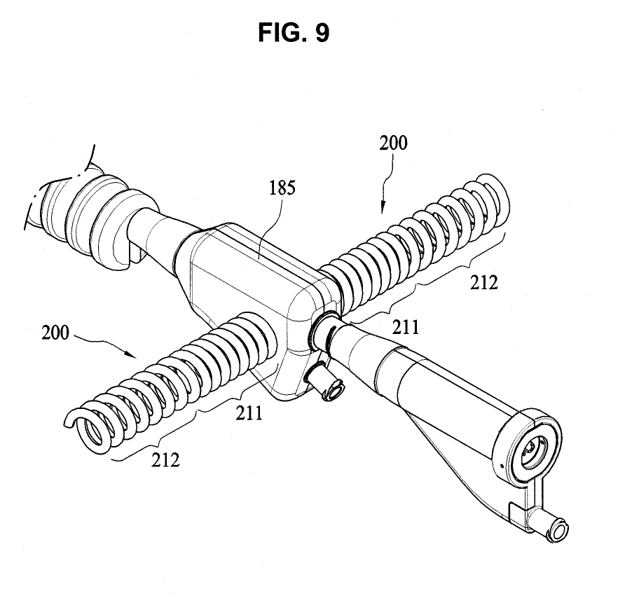

As shown in FIG. 9, the present invention may further include a handle 200. The handles may be provided at both sides of the first housing 185, and even though not shown, the handles may be provided at both sides of the second housing 195.

In this case, the user may perform surgery in a very stable posture by holding the tissue dissector 100 of the present invention with both hands.

In addition, when the handle 200 is configured so that one end 210 fixed to the second housing 195 has rigidity and the other end 220 forming a free end has elasticity, the vibration transmitted from the driving motor 160 to the wrist is alleviated to minimize the fatigue felt in the long-term use.

The handle 200 is preferably formed in a coil shape to facilitate gripping.

On the other hand, when the abnormal tissue to be dissected during surgery is located at a position difficult to cut, such as a back of the obstacle or a groove, the outer tube 110 is inserted after bending the inner tube 120 so that the cutting head 130 may be easily approached to the target abnormal tissue (see FIG. 6). The cutting head 130 of the present invention is connected to the driving shaft 150 through the cable 140 to be normally rotated even when the inner tube 120 is bent. Therefore, it is possible to cut very precisely the abnormal tissue at an optimum angle by approaching as close as possible to the target abnormal tissue.

The bending of the inner tube 120 needs to be performed during surgery in some cases during surgery. For this purpose, a bending device 500 for the tissue dissector of the present invention is configured as follows.

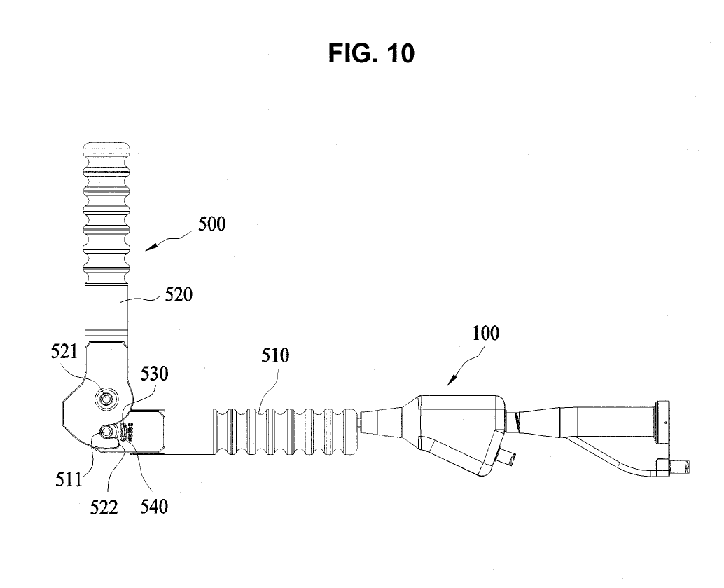

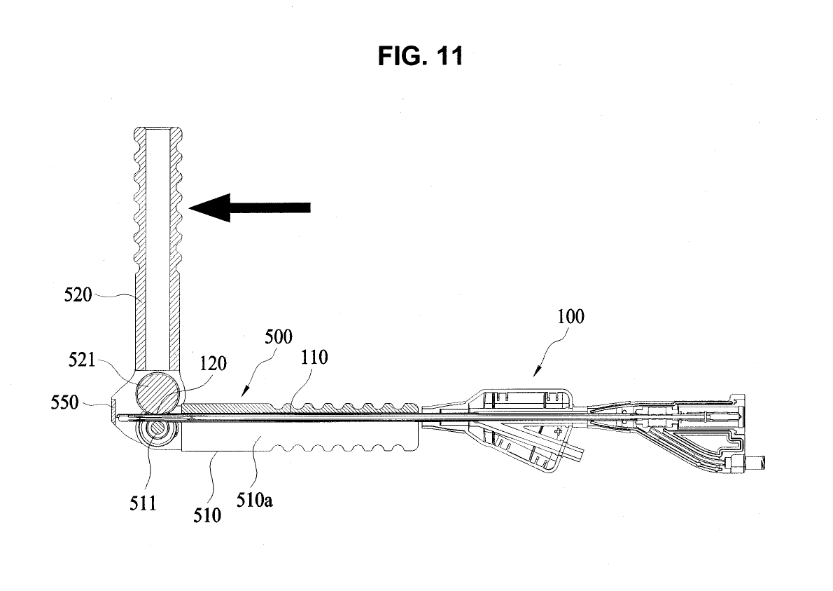

As shown in FIGS. 10 and 11, the bending device 500 for the tissue dissector of the present invention includes a fitting portion 510 into which the outer tube 110 of the tissue dissector 100 is inserted, and a rotation lever 520 rotatably coupled to a rear end of the fitting portion 510.

In addition, a first roller 511 is fixed to the rear end of the fitting portion 510. The first roller 511 is in close contact with the inner tube 120 protruding from the front end of the outer tube 110.

In addition, a second roller 521 facing the first roller 511 is fixed to the second housing 195 with the inner tube 120 interposed therebetween.

The fitting portion 510 is formed with an opening 510a in a longitudinal direction across the entire space. The opening 510a is formed in a bending direction (downward in the drawing) on the same plane as the first roller 511 so that the outer tube 510 can be separated while being fitted to the fitting portion 510 through the opening 510a.

Meanwhile, a plurality of angle adjusting grooves 530 are provided on one side of the fitting part 510 along a rotation path of the rotation lever 520. An angle adjusting pin 540 may be coupled to the angle adjusting groove 530 to restrict the rotation angle of the rotation lever 520 by interfering with the end 522 of the rotation lever 520.

In this case, the rotation angle of the rotation lever 520 varies according to the coupling position of the angle adjusting pin 540, so that the user may accurately bend the inner tube 120 by a desired angle.

In addition, when the outer tube 110 is fitted too deeply to the fitting portion 510, an accident may occur that the outer tube 110 is positioned between the first roller 511 and the second roller 521. As a result, a shielding piece 550 for preventing the inner tube 120 from moving forward beyond a predetermined position may be provided at one side of the rotation lever 520.

In this case, if the user merely inserts the outer tube 110 into the fitting portion 510 to the end until the front end of the outer tube 110 comes into contact with the shielding piece 550, the outer tube 110 is always located between the first roller 511 and the second roller 521, so that it is possible to perform very easily a precise bending operation.

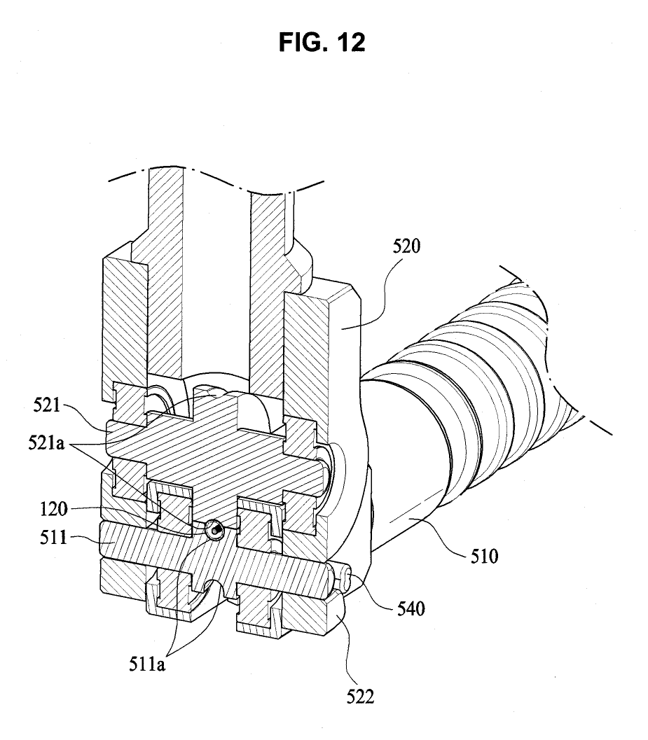

As shown in FIG. 12, it is preferable that semicircular grooves 511a and 521a are formed on the outer circumferential surfaces of the first roller 511 and the second roller 521 to maximize the adhesion with the inner tube 120.

In this case, due to the uniform external force applied to the inner tube 120 by the grooves 511a and 521a across the area during bending, the inner tube 120 is bent stably without being separated from the rollers 511 and 521 during bending and deformation of the cross section does not occur.

When the groove 511a of the first roller 511 is formed to be deeper than the radius of the inner tube 120, the bending is performed more stably while the seating performance of the inner tube 120 with respect to the first roller 511 is greatly improved, and the deformation of the cross section may be minimized.

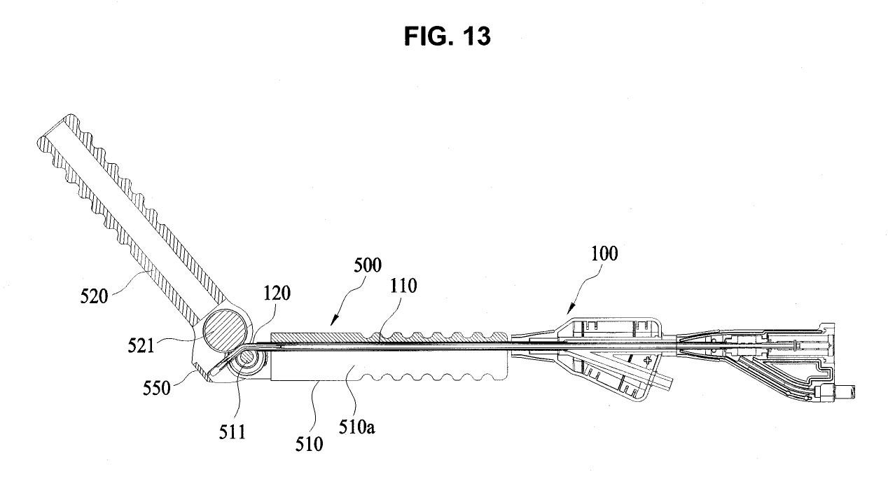

When the rotation lever 520 rotates downward after fitting the outer tube 110 of the tissue dissector 100 through the opening 510a of the fitting portion 510 so that the inner tube 120 is located between the first roller 511 and the second roller 521, the bending device 500 of the present invention may easily bend the inner tube 120 without disassembling the tissue dissector 100 because the inner tube 120 is bent while the second roller 521 rotates together with the rotation lever 520 based on the first roller 511 as shown in FIG. 13.

When the tissue dissector 100 is rotated in the direction of the opening 510a of the fitting part 510 in a state where the inner tube 120 is bent, the inner tube 120 bent together with the outer tube 110 is discharged through the opening 510a and the tissue dissector 100 is easily separated from the bending device 500.

Accordingly, when the bending device of the present invention is used, the tissue dissector 100 can be freely used immediately according to the surgery situation while bending the inner tube 120, thereby greatly improving the accuracy of the operation.

As described above, those skilled in the art to which the present invention belongs will be able to understand that the present invention can be implemented in other detailed forms without changing the technical spirit or an essential characteristic. Therefore, it should be understood that the aforementioned exemplary embodiments are just illustrative and not restrictive in all aspects. The scope of the present invention is defined by not the specification, but the following claims, and all of changes and modifications obtained from the meaning and range of claims and equivalent concepts should be construed as being included in the scope of the present invention.

### EXPLANATION OF REFERENCE NUMERALS AND SYMBOLS

| | |
|---|---|
| 100: Tissue dissector | 110: Outer tube |
| 120: Inner tube | 130: Cutting head |
| 140: Cable | 150: Driving shaft |
| 160: Driving motor | 180: Suction tube |
| 190: Fluid supply tube | 195: Second housing |
| 500: Bending device | 510: Fitting portion |
| 520: Rotary lever | 511: First roller |
| 521: Second roller | |

What is claimed is:

1. An assembly for dissecting a tissue comprising: a tissue dissector and a bending device,
wherein the tissue dissector includes
an outer tube inserted into the human body;
an inner tube inserted into the outer tube so that a front end protrudes from a front end of the outer tube;
a cutting head located at the front end of the inner tube;
a cable connected to a rear end of the cutting head;
a driving shaft connected to a rear end of the cable; and
a drive motor for applying a rotational force to the driving shaft,
wherein the bending device includes:
a fitting portion to which the outer tube is fitted;
a rotation lever rotatably coupled to a rear end of the fitting portion;
a first roller fixed to an end of the fitting portion so as to be in close contact with a front end of the inner tube;
a second roller which is fixed to the rotation lever so as to face the first roller with the inner tube interposed therebetween and applies a bending force to the inner tube while rotating about the first roller when the rotation lever rotates;
an angle adjusting groove provided on a rotation path of the end of the rotation lever when the rotation lever is rotated to one side of the fitting portion; and
an angle adjusting pin which is provided in the angle adjusting groove so as to interfere with an end of the rotating rotation lever so as to restrict a rotation angle of the rotation lever,
wherein a semicircular groove for maximizing the adhesion with the inner tube is provided on an outer circumferential surface of the first roller and the second roller.

2. The assembly for dissecting a tissue of claim 1, further comprising:
a suction tube connected to a rear end of the outer tube so as to collect the dissected tissue by introducing the tissue dissected by the cutting head through the front end of the outer tube, and
a fluid supply tube connected to the rear end of the outer tube to discharge a fluid to the front end of the outer tube.

3. The assembly for dissecting a tissue of claim 2, further comprising:
a closed type second housing which accommodates a rear end of the inner tube passing through the rear end of the outer tube, a rear end of the driving shaft passing through the rear end of the inner tube, and the rotation shaft of the driving motor,
wherein the fluid supply tube and the suction tube non-detachably communicate with the outer surface inside the sealed second housing.
